(19) United States
(12) Patent Application Publication
(10) Pub. No.: US 2011/0301233 A1
(43) Pub. Date: Dec. 8, 2011

(54) Novel Polyisoprenylated Benzophenone Derivatives from Garcinia paucinervis

(76) Inventors: XUE-MEI GAO, Hong Kong (CN); Ting Yu, Singapore (SG); Kathy Qian Luo, Singapore (SG); Hong Xi Xu, Hong Kong (CN)

(21) Appl. No.: 12/793,691

(22) Filed: Jun. 4, 2010

(51) Int. Cl. A61K 31/343; C07D 307/77; A61P 35/00; C07D 307/94

(52) U.S. Cl. 514/462; 549/331; 549/459; 514/468

(57) ABSTRACT

Novel polyisoprenylated benzophenone derivatives isolated from the plant Garcinia paucinervis. Among these new compounds, paucinones A-C (Compounds 1-3) contains an unexpected cyclohexane-spiro-tetrahydrofuran moiety. A 1-methylene-3,3-dimethylcyclohexane group never reported before was found in the structure of paucinone D (Compound 4). Structures of these compounds were elucidated with spectroscopic evidence. The relative stereochemistry of 1-4 was determined by NOESY correlations. These compounds a potent ability to activate caspase-3 in HeLa-C3 cells within 72 hours at a low concentration and significant cytotoxicity against HeLa-C3 cells.

### FIELD OF THE INVENTION

This invention relates to new chemical entity isolated from natural sources for their therapeutic uses. More particularly, it relates to a compound that is naturally occurring in the plant of Garcinia paucinervis and its biological activity of inducing apoptosis of tumor cells and inhibitory effect on tumor cell growth.

### BACKGROUND OF THE INVENTION

Apoptosis is a genetically programmed and physiological mode of cell death that leads to the removal of unwanted or abnormal cells. Effective cancer therapeutic strategies often rely on preferential and efficient induction of apoptosis in tumor cells. Caspase-3 protease plays important roles in the signaling pathway controlling mammalian apoptosis. Natural products which can activate caspase-3 are functionally important in the induction of apoptosis and represent a type of bioactive natural products.

While with today's high throughput chemical synthetic technologies and high efficiency screening methodologies it may be easy to find a large number of chemical compounds that shows promising biological effect at cellular levels in the laboratory, it appears that the rate of these compounds becoming clinically useful is very low due to a number of factors. One of the factors is their toxicity, which are often found to be to too serious to be tolerable by human body at a later stage of the new drug development. In this respect, new compounds discovered from natural sources which have been used as medicines for thousand years are believed to hold advantages because they have been consumed by human for a long time and their toxicity therefore are more likely to be tolerable than purely synthetic compounds.

### SUMMARY OF THE INVENTION

The tropical genus Garcinia is well known to be a rich source of bioactive isoprenylated xanthones and benzophenones. The present invention is part of the continuing effort for finding new bioactive compounds in this genus. Four novel polyisoprenylated benzophenone derivatives, paucinones A-D (compounds 1-4), were isolated from Garcinia paucinervis. Their isolation and structure elucidation as well as their cytotoxicity against Hela-C3 cells are disclosed herewith.

One object of the present invention is to provide new compounds having the effect of inducing apoptosis and are useful for developing into anti-cancer drugs. This objective was achieved by isolating four novel polyisoprenylated benzophenone derivatives, paucinones A-D (Compounds 1-4) from Garcinia paucinervis that have such effect on apoptosis. The novel compounds' structural are shown in FIGS. 3 and 4.

The various features of novelty which characterize the invention are pointed out with particularity in the claims annexed to and forming a part of this disclosure. For a better understanding of the invention, its operating advantages, and specific objects attained by its use, reference should be made to the drawings and the following description in which there are illustrated and described preferred embodiments of the invention.

### BRIEF DESCRIPTION OF THE DRAWINGS

FIG. 1 depicts selected HMBC (→) and ¹H-¹H COSY (−) correlations between Compounds 1 and 4;

FIG. 2 shows the key NOESY correlations and relative configurations assigned for Compounds 1 and 4 and their corresponding interatomic distance [A];

FIG. 3 shows a possible biosynthesis pathway of paucinones A-C (1-3);

FIG. 4 shows a possible biosynthesis pathway of paucinone D (4);

FIG. 5 shows morphology changes during the course of compound treatment;

FIG. 6 shows ¹H and ¹³C NMR data for paucinones A-D (Compounds 1-4) in CD₃OD.

### DETAILED DESCRIPTION OF PARTICULAR EMBODIMENTS OF THE INVENTION

### Compound Isolation

An acetone extract prepared from the leaves of Garcinia paucinervis(2.8 kg) was partitioned between H₂O and CH₂Cl₂. The CH₂Cl₂-soluble portion (182 g) was decolourized by MCI. The 90% methanol portion (57 g) was chromatographed on a silica gel column eluting with hexane-acetone (1:0, 4:1, 2:1, 1:1 and 0:1) to afford five fractions, I-V. 7 grams of Fraction II were then separated on reversed-phase column (RP-18) eluting with MeOH/H₂O (80%-100%) to give 17 fractions. Fraction II-1-10 was separated over Sephadex LH-20 eluting with MeOH and then subjected to semi-preparative HPLC (MeOH—H₂O, 80:20) to yield paucinone A (compound 1, 2.1 mg), paucinone B (compound 2, 2.0 mg), paucinone C (compound 3, 3.4 mg), and paucinone D (compound 4, 2.1 mg).

### Compound Characterization

Compounds 1-4 shared several common spectral characteristics. The UV spectra showed absorption bands consistent with those of aromatic rings and conjugated carbonyl groups. The IR spectra exhibited bands for hydroxyl groups, conjugated carbonyl groups and aromatic rings.

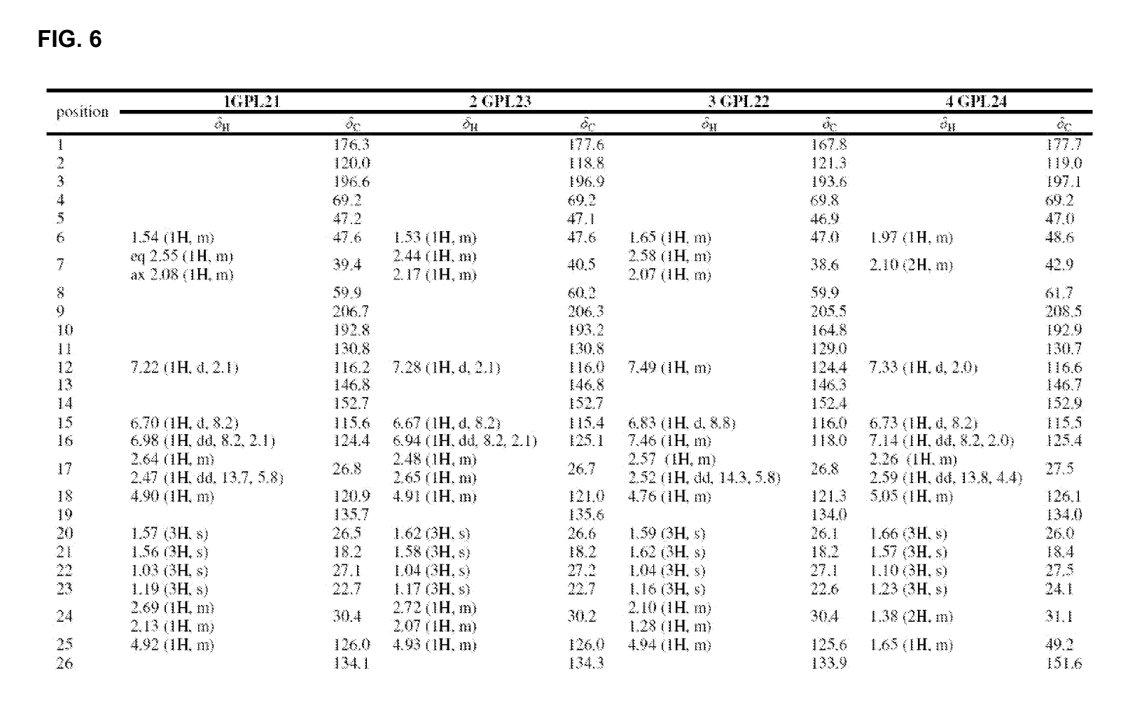

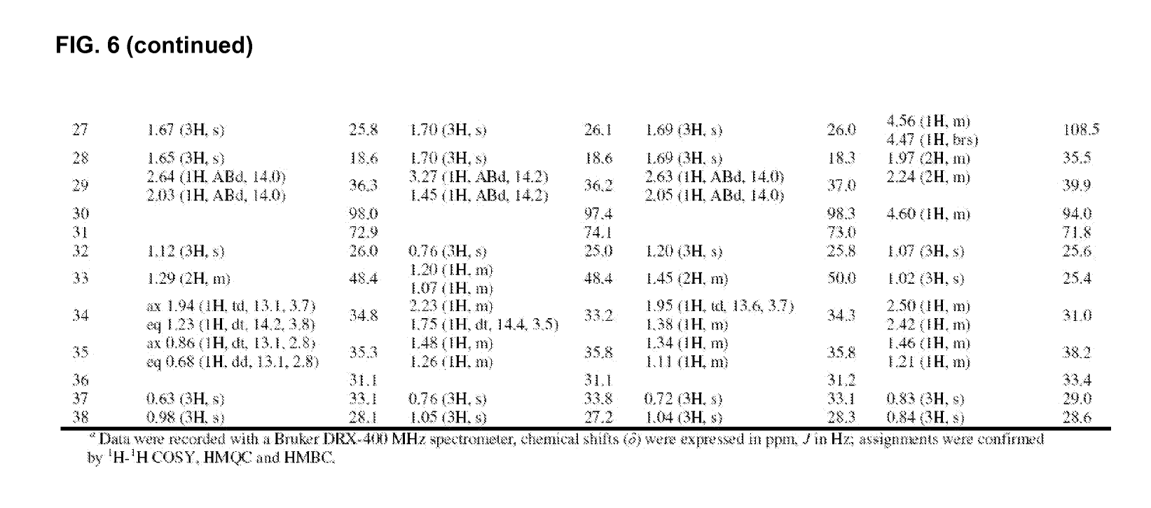

Paucinone A (Compound 1) was obtained as a white powder. Its molecular formula was established as C₃₈H₅₀O₇ by HRESIMS at m/z 619.3624 [M+H]⁺, suggesting fourteen degrees of unsaturation. The ¹H and ¹³C NMR data of Compound 1 (FIG. 6) showed the presence of nine methyls, seven methylenes, six methines (five olefinic), and sixteen quaternary, carbons (seven olefinic, one oxygenated, and three carbonyls). In addition, the IR spectrum showed the presence of hydroxyl groups (3435 cm⁻¹), carbonyl groups (1733 cm⁻¹) and aromatic rings (1606 cm⁻¹). The analysis of 2D NMR spectra using HMQC and HMBC techniques enabled the assignment of ¹H and ¹³C NMR signals. The foregoing data indicated that 1 was a benzophenone derivative that contained four isoprene units.

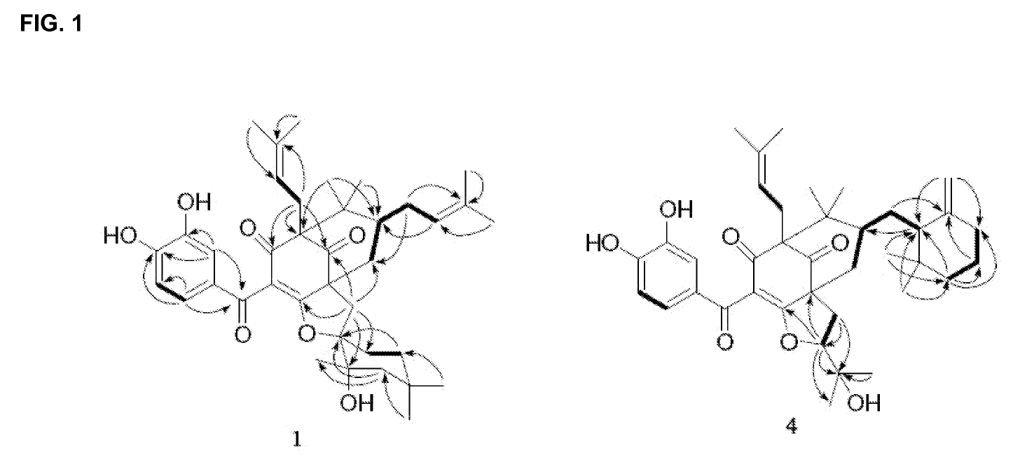

Since the NMR data of Compound 1 were similar to those of the known coccinone A, the possible structure was established by a detailed comparison of its NMR data with those of this known compound, and suggesting the same core structure of both compounds. However, different carbon and proton chemical shifts for C-29, C-30, and C-31 indicated that the structure of 1 differed from coccinone A with respect to the side chain attached at C-1 and C-8. In the HMBC spectrum of 1, the correlations of the proton signals at δ_{H} 2.64 and 2.03 (H₂-29) with the carbon signals at δ_{C} 98.0 (C-30) and 72.9 (C-31), and with the carbon signals at δ_{C} 176.3 (C-1), 39.4 (C-7), 59.9 (C-8), and 206.7 (C-9), suggested that an oxygen bridge was formed between C-30 and C-1. A cyclohexane ring connected with the tetrahydrofuran ring through C-30 was elucidated by the HMBC correlations of H₂-33 (δ_{H} 1.28-1.30) with C-30 (δ_{C} 98.0, s), C-31 (δ_{C} 72.9, s), C-34 (δ_{C} 34.8, t), C-35 (δ_{C} 35.3, t), and C-36 (δ_{C} 31.1, s), of H₂-34 (δ_{H} 1.94, 1.23) with C-29 (δ_{C} 36.3, t), C-30, C-31, C-35, and C-36, and of H₂-35 (δ_{H} 0.86, 0.68) with C-30, C-33 (δ_{C} 48.4, t), C-34, and C-36 (see FIG. 1). The above deduction revealed that Compound 1 contained a cyclohexane-spirotetrahydrofuran group. The HMBC correlation of H₃-32 (δ_{H} 1.12) with C-30, C-31 and C-33, together with the molecular formula C₃₈H₅₀O₇ indicated the presence of a methyl and a hydroxyl group at C-31. A gem-dimethyl group at C-36 was deduced by the HMBC correlations of H₃-37 (δ_{H} 0.63) and H₃-38 (δ_{H} 0.98) with C-33, C-35 and C-36. Finally, the key information was again provided by the correlations between protons H₂-29 and the two sets of carbons, on one hand with carbons at C-1, C-7, C-8 and C-9 of the benzophenone moiety and on the other with C-30, C-31 and C-34 of the cyclohexane ring.

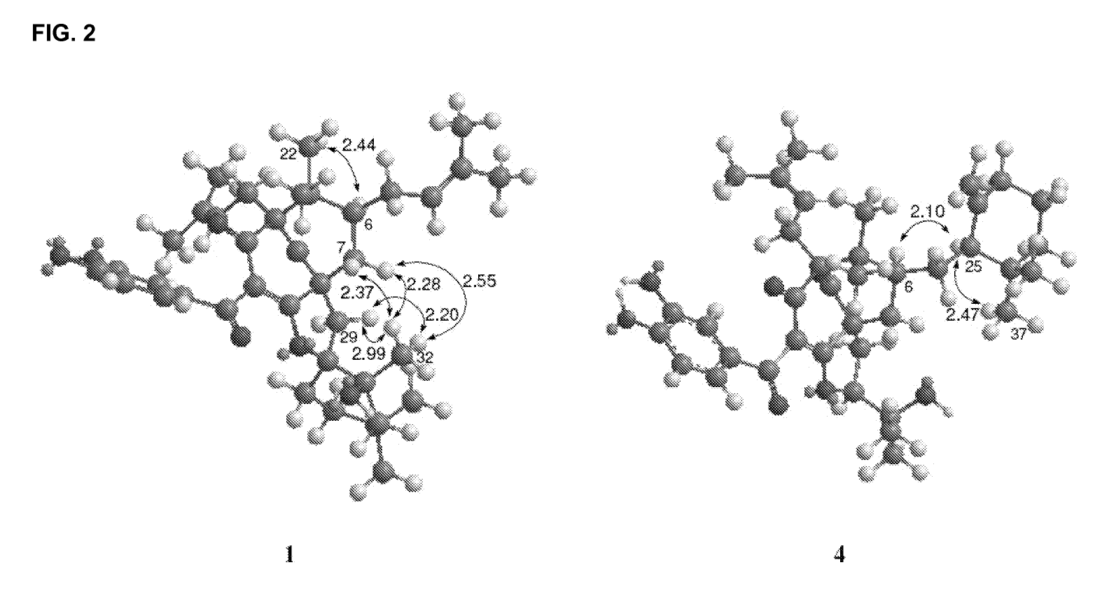

The relative configuration of Compound 1 was revealed by an NOE experiment. The NOESY correlation of H-61H₃-22 suggested the existence of equatorial prenyl group at C-6. (FIG. 2) This was confirmed by the ¹³C NMR chemical shift of Me-22, which was due to the γ-gauche interaction shielding of the axial methyl by the C-6 substituent when this group is equatorial.

The axial or equatorial positions of the cyclohexanic protons were assigned by taking into account the coupling constant values and the NOESY correlations (see FIG. 2). The NOESY correlations of H-35ax (δ_{H} 0.86) with H₃-37 (δ_{H} 0.63) and of H-34ax (δ_{H} 1.94) with H₃-38 (δ_{H} 0.98) suggested the axial position of both hydrogens. Conversely, protons at δ_{H} 1.23 and 0.68 were in equatorial position (H-34 eq and H-35 eq). These assignments were confirmed by the coupling constant³ J=13.1 Hz between H-34ax and H-35ax, characteristic of an anticoplanar stereochemistry. Finally, the strong NOESY correlation of H₃-32 (δ_{H} 1.12) with H-29b (δ_{H} 2.03) and H-7 eq (δ_{H} 2.53-2.56) led to the determination of the equatorial position of Me-32 and conversely the axial position of hydroxyl group. From these spectroscopic data, the structure of Compound 1 is determined as shown.

Paucinone B (Compound 2) was isolated as a white amorphous solid. The molecular formula of Compound 2 was determined to be C₃₈H₅₀O₇ by HRESIMS at m/z 619.3624 [M+H]⁺, which was the same as that of 1. Comparison of the NMR data between Compound 2 and Compound 1 indicated that they are isomers (FIG. 6). The only structural difference between Compound 2 and Compound 1 was found to be the opposite configuration of Me-32. This was deduced from the chemical shifts and the NOESY correlations. The ¹³C NMR chemical shift of C-31 was at δ_{C} 74.1 in Compound 2, while the signal of C-31 with an equatorial position was located at δ_{C} 72.9 in Compound 1. There were no correlation of H₃-32 with H-7 eq and very weak correlation of H-32 with H-29b in the NOESY spectrum of Compound 2. These observations led to the determination of the axial Me-32 and conversely the equatorial hydroxyl group. Comparing to Compound 1, the above deduction was confirmed by the upfielded chemical shifts of H-29b (δ_{H} 1.45), H₃-32 (δ_{H} 0.76), and H₂-33 (δ_{H} 1.20, 1.07), and downfielded chemical shifts of H-29a (δ_{H} 3.27), H₂-34 (δ_{H} 2.23, 1.75), H₂-35 (δ_{H} 1.48, 1.26), H₃-37 (δ_{H} 0.76) and H₃-38 (δ_{H} 1.05) due to gauche effect. Therefore, the structure of Compound 2 was established as an isomer of Compound 1 as shown.

Paucinone C (Compound 3) was obtained as a white amorphous solid. The HRESIMS showed an ion peak at m/z 635.3580 [M+H]⁺, giving the molecular formula C₃₈H₅₀O₈. The NMR data of Compound 3 were similar to those of Compound 1 and Compound 2 indicating that the three compounds have the same carbon skeleton. In contrast, the only difference between them was that there was one more oxygen atom in Compound 3 than in Compound 1 and Compound 2. The NMR spectra of Compound 3 showed difference at C-10 when compared with those of Compound 1 and Compound 2. The chemical shift of C-10 was upfielded to δ_{C} 164.8 instead of δ_{C} 192.8 and 193.2 in Compound 1 and Compound 2, respectively. Within the given molecular formula C₃₈H₅₀O₈, an ester group was found to locate between C-2 and C-11, which has never been reported among publicly disclosed benzophenone analogues. According to the observed ROESY correlations and comparison of ¹H and ¹³C NMR data with those of Compound 1 and Compound 2 (Table 1), the relative configuration of 3 was deduced as being the same as that of Compound 1 with equatorial Me-32 at C-31. Consequently, the structure of Compound 3 was established as shown.

Paucinone D (Compound 4), obtained as a white amorphous solid, gave the molecular formula C₃₈H₅₀O₇, as revealed by its HRESIMS at m/z 619.3625 [M+H]⁺. The ¹H and ¹³C NMR data for Compound 4 were similar to those of Compound 1 with differences in substituents at C-30 and C-24. The COSY and HMBC spectra suggested the presence of a 1 methylene-3,3-dimethylcyclohexane at C-24 (FIG. 1). In the HMBC spectrum, the presence of a gemdimethyl group was deduced from the correlations of the methyl protons H₃-37 (δ_{H} 0.83) and H₃-38 (δ_{H} 0.84) with the quaternary carbon C-36 (δ_{C} 33.4), the methylene carbon C-35 (δ_{C} 38.2) and the methine C-25 (δ_{C} 49.2). Correlations were also observed from C-28 (δ_{C} 35.5) and C-25 to a characteristic exocyclic methylene protons H₂-27 (δ_{H} 4.47 and 4.56-4.60), from C-25, C-26 (δ_{C} 151.6) and C-36 to the methylene protons H₂-24 (δ_{H} 1.36-1.40), and from C-26, C-28, C-35, and C-36 to H₂-34 (δ_{H} 2.40-2.44 and 2.48-2.53), which confirmed the presence of 1-methylene-3,3-dimethylcyclohexane moiety located at C-24. HMBC correlations of H₂-29 with C-1, C-8, C-9, C-30 and C-31 suggested an oxygen bridge between C-30 and C-1. The HMBC correlations of H-30 with C-31, C-32, and C-33 (FIG. 1), and of H₃-32 and H₃-33 with C-30 and C-31 suggested an iso-propyl group at C-30. The carbon signal of C-31 (δ_{C} 71.8, s) together with molecular formula C38H50O7 indicated the presence of a hydroxyl group at C-31.

In the NOESY spectrum, the correlation of H-25 (δ_{H} 1.65) with H-6 (δ_{H} 1.98) and H₃-37 (δ_{H} 0.83) suggested an α-orientation for H-25. The absence of correlation between H 30 and H₂-7 indicated the α-orientation of H-30. (FIG. 2) These data, together with other results from 2D NMR analysis confirmed the structure of Compound 4.

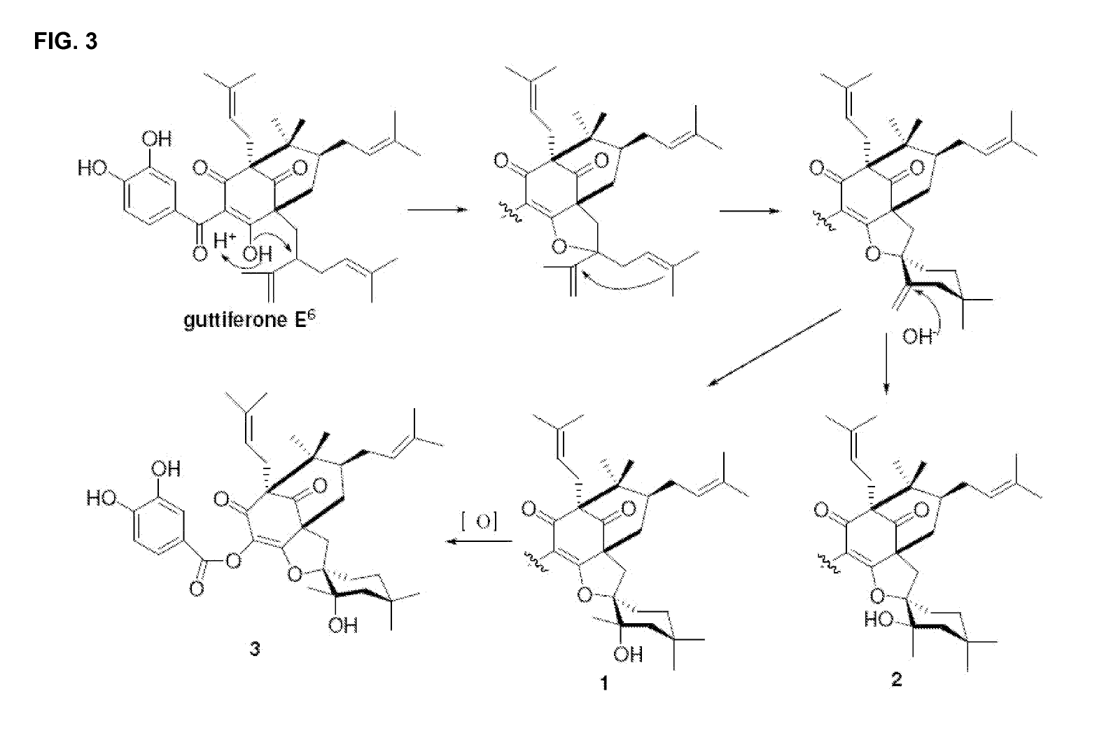

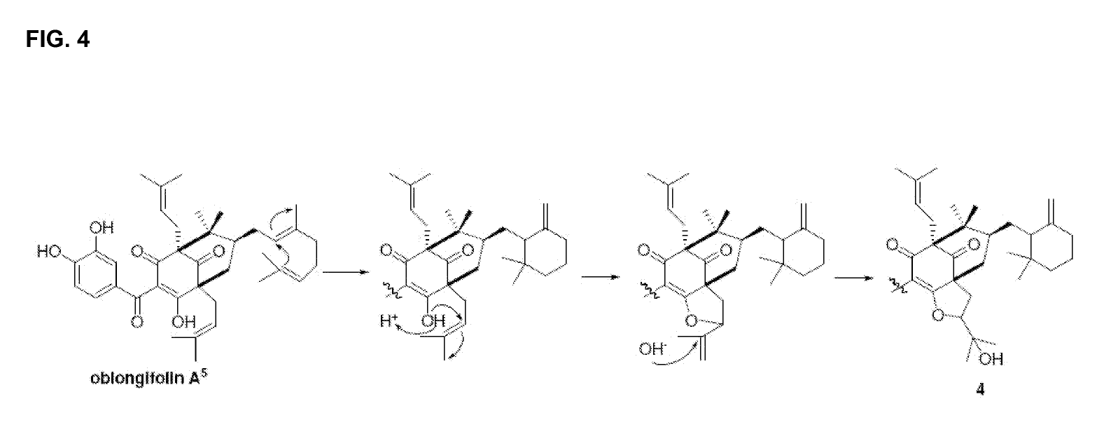

The cyclohexane-spiro-tetrahydrofuran moiety of Compounds 1-3 and the 1-methylene-3,3-dimethylcyclohexane moiety of Compound 4 shed new insights into structural diversity of benzophenone analog libraries. The possible biosynthesis pathways of these four new benzophenones are given in FIG. 3 and FIG. 4.

### Biological Activity

The biological activity of Compounds 1-4 was evaluated for apoptosis-inducing effects using genetically engineered HeLa-C3 cells that can produce a fluorescent biosensor capable of detecting caspase-3 activation. These cells emit a green light under normal growth conditions and change to a blue light when caspase-3 is activated during apoptosis to cleave the sensor protein inside the cells. This color change allows one to use a fluorescent plate reader to directly detect the activation level of caspase-3 in HeLa-C3 cells during the course of the compound treatment in a noninvasive way. Based on our previous test results, the emission ratio of YFP (yellow fluorescent protein)/CFP (cyanfluorescent proteins) is usually between 6 and 8 in normal cells, and this ratio will decrease to a value below 3 if a compound can activate caspase-3 and kill cancer cells. Therefore, any compound that can reduce the YFP/CFP emission ratio to a value below 3 is considered positive in activating apoptosis.

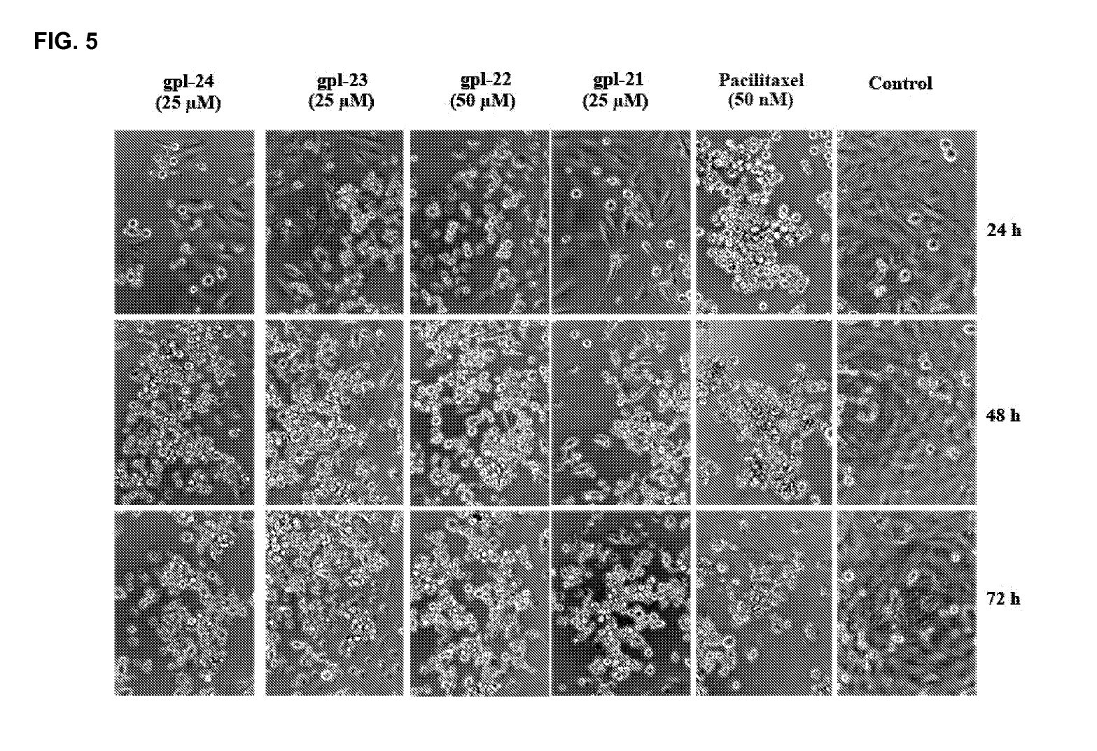

As shown in FIG. 5, HeLa-C3 cells were treated with Compound 1 (gpl21) at 25 μM, Compound 3 (gpl22) at 50±2M, Compound 2 (gpl 23) at 25 μM, Compound 4 (gpl24) at 25 μM, an anticancer drug, paclitaxel at 500 nM (serving as a positive control), or without any drug (control) for 24, 48, and 72 h. Various cell morphologies were recorded at the indicated time points. The control cells have normal attached cell morphology through the course of the experiment. Cells have shrinkage and detached morphology when they were treated with either the anticancer drug paclitaxel or other compounds, confirming their ability to kill cancer cells.

As shown in Table 1, compounds were tested at a concentration of 100 μM, 50 μM and 25 μM. 1, 2 and 4 were found to reduce the YFP/CFP emission ratio below 3 within 72 h at these three concentrations. Compound 3 could activate caspase-3 in HeLa-C3 cells within 72 h at 100 μM and 50 μM.

TABLE 1
Apoptosis-Inducing Effects at 72 h

| | Apoptotic effect at | | |
|---|---|---|---|
| compound | 100 μM | 50 μM | 25 μM |
| 1 | + | + | + |
| 2 | + | + | + |
| 3 | + | + | − |
| 4 | + | + | + |

“+” means the YFP/CFP emission ratio of compound treated HeLa-C3 cells was below 3 at 72 h. “−” means the YFP/CFP emission ratio of compound treated HeLa-C3 cells was above 3 at 72 h.

In addition to detecting the caspase activation using our HeLa-C3 cells, the cell morphology changes was also examined after the treatment of the tested compounds. As shown in FIG. 5, in the control sample, with no compound treatment, the cells attached to the culturing surface with normal cell morphology. When cells were treated with a clinically used anticancer drug, paclitaxel (500 nM), which is known to induce apoptotic cell death, the cells initially rounded up and then appeared with typical cell shrinkage morphology. Similar cell shrinkage was observed in HeLa-C3 cells treated with Compound 1, Compounds 2 and 4 at 25 μM and Compound 3 at 50 μM. Since both caspase activation and cell shrinkage are landmark events only occurring in apoptotic cells, it is concluded that these tested compounds are capable of inducing apoptotic cell death in HeLa-C3 cells.

TABLE 2
IC50 values of HeLa cells treated
with different compounds for 72 h

| Compound | IC₅₀ (μM) |
|---|---|
| 1 | 10 ± 0.5 |
| 2 | 8.2 ± 0.8 |
| 3 | 24.3 ± 0.6 |
| 4 | 5.8 ± 0.6 |

To determine the cytotoxicity of four compounds, their IC₅₀ on HeLa cells was measured. As shown in the Table 2, the Compound 1, Compound 2 and Compound 4 have strong HeLa cell growth inhibiting effects, since their IC₅₀ are below or near 10

### Experimental Details

General Experimental Procedures. Optical rotations were measured with a JASCO DIP-1000 polarimeter. Ultraviolet absorption spectra were recorded using a Perkin-Elmer Lambda L14 spectrometer. A Perkin Elmer spectrum 100 FT-IR spectrometer was used for scanning IR spectroscopy with KBr pellets. 1D and 2D NMR spectra were recorded on a Bruker AV-400 spectrometer with TMS as internal standard. Chemical shifts (δ) were expressed in ppm with reference to the solvent signals. HRMS were obtained using a nanoLC-MS/MS system, with a nanoAcquity HPLC module and a Q-TOF spectrometer equipped with a nanoelectrospray ion source (Waters, Milford, Mass.) and supported by a lock-mass apparatus to perform a real-time calibration correction. Column chromatography was performed with silica gel (200-300 mesh, Qingdao Marine Chemical, Inc., Qingdao, People's Republic of China), Sephadex LH-20 (Pharmacia), and reversed-phase C18 silica gel (250 mesh, Merck). Precoated TLC sheets of silica gel 60 GF254 were used. An Agilent 1100 series equipped with an Alltima C18 column (4.6×250 mm) was used for HPLC analysis, and semipreparative and preparative Alltima C18 columns or Zorbax SB-C18 columns (9.4×250 mm and 22×250 mm) were used in sample preparation. Spots were visualized by heating silica gel plates sprayed with 10% H₂SO₄ in EtOH.

Plant Material. The stems of G. paucinervis were collected in October 2008 from Xishuangbanna Prefecture of Yunnan Province, China. The plant was identified by Pan-Yu Ren.

Paucinone A (Compound 1): white amorphous powder; [a]²³_{D}-6.2 (c 0.05, MeOH); UV (MeOH) λₘₐₓ (log ε) 273 (2.30), 234 (2.32), 203 (2.50) nm; IR (KBr) nₘₐₓ 3435, 2924, 1733, 1606, 1442, 1384, 1293, 1202, 1109, 1064, 997, 957 cm⁻¹; ¹H and ¹³C NMR data, Table 1; positive HRESIMS m/z 619.3624 [M+H]⁺ (calcd 619.3635 for C₃₈H₅₁O₇).

Paucinone B (Compound 2): white amorphous solid; [α]²⁵_{D} +58.7 (c 0.10, MeOH); UV (MeOH)λₘₐₓ (log ε) 275 (2.33), 234 (2.32) nm; IR (KBr)νₘₐₓ 3436, 2925, 1735, 1608, 1519, 1442, 1372, 1294, 1194, 1108, 990, 959 cm⁻¹; ¹H and ¹³C NMR data, Table 1; positive HRESIMS m/z 619.3624 [M+H]⁺ (calcd 619.3635 for C₃₈H₅₁O₇).

Paucinone C (Compound 3): white amorphous solid; [a]²⁴_{D} +19.2 (c 0.17, MeOH); UV (MeOH)νₘₐₓ (log δ) 268 (2.41), 222 (2.41) nm; IR (KBr) νₘₐₓ 3435, 2926, 1741, 1631, 1444, 1367, 1292, 1200, 1100, 1066, 1025 cm⁻¹; ¹H and ¹³C NMR data, Table 1; positive HRESIMS m/z 635.3580 [M+H]⁺ (calcd 635.3584 for C₃₈H₅₁O₈).

Paucinone D (Compound 4): white amorphous solid; [a]²⁷_{D} +41.6 (c 0.11, MeOH); UV (MeOH) νₘₐₓ (log ε) 274 (2.40), 251 (2.22) nm; IR (KBr) νₘₐₓ 3435, 2924, 1732, 1630, 1443, 1375, 1291, 1199, 1114, 979 cm⁻¹; ¹H and ¹³C NMR data, Table 1; positive HRESIMS m/z 619.3625 [M+H]⁺ (calcd 619.3635 for C₃₈H₅₁O₇).

Bioassay: The bioassay method was described in our previous paper with some modifications. All the testing samples were dissolved in DMSO to make stock solutions. The concentration of each stock was at least 1000 times higher than the working concentration. HeLa-C3 cells, which can detect apoptotic cell death involving caspase activation, were cultured in minimum essential medium (MEM) containing 10% fetal bovine serum, 100 U/ml penicillin, 100 mg/ml streptomycin, in a 5% CO2 humidity incubator at 37° C. The sample well for apoptotic activity testing was prepared by seeding a well on a 96-well plate with 7500 HeLa-C3 cells suspended in 100 μl culture medium. After 12-16 h incubation, the old medium was removed and 100 μl freshly prepared culture medium containing the testing sample at a certain working concentration was added to both the sample well and the corresponding background well. Culture medium containing 0.1% DMSO was the negative control while 500 nM paclitaxel was the positive control. After that, the plate was read repeatedly by a Perkin-Elmer Victor reader with excitation wavelength at 440±10 nm and emission wavelength at 486±8 nm for CFP (cyan fluorescent protein) and 535±8 nm for YFP (yellow fluorescent protein) at indicated time points. The data acquisition duration was up to 72 h. The YFP/CFP emission ratio was then calculated. The background fluorescence was measured from the wells containing only medium. After subtracting the background fluorescence from the recorded signal, net YFP and CFP readings were obtained. In this paper, Y/C emission ratio is used to represent the effect of FRET, which is equal to the net YFP reading divided by the net CFP reading from the same well. If YFP/CFP emission ratio was reduced below 3, the testing sample was considered as a good apoptotic inducer at that concentration. All samples were tested in triplicate. The whole experiment was repeated for three times.

IC₅₀ of four new compounds was measured using MTT assay. MTT powder was dissolved in PBS at a concentration of 5 mg/mL. For MTT assay, 10 μL of MTT solution was added into each well of a 96-well plate. After 2 h incubation at 37° C., 100 μL 10% SDS solution with 0.01 M HCl was added to dissolve the purple crystals. After 24 h incubation, the optical density (OD) readings at 595 nm were measured using a plate reader. Firstly, 2,500 HeLa cells suspended in 100 μL MEM medium were seeded respectively in a 96-well plate. After 24 h incubation, fresh medium that contained various concentrations of each compound were added into the 96-well plate and changed the old medium. The concentrations applied were ranged from 100 μM to 1.5625 μM, which was achieved by doing two-fold dilutions for 6 times. The OD values of the control group at 0 h and 72 h together with the compound treated groups at 72 h from the MTT assay were measured using a plate reader. IC₅₀ is the concentration of a compound inhibiting 50% of the cell growth.

While there have been described and pointed out fundamental novel features of the invention as applied to a preferred embodiment thereof, it will be understood that various omissions and substitutions and changes, in the form and details of the embodiments illustrated, may be made by those skilled in the art without departing from the spirit of the invention. The invention is not limited by the embodiments described above which are presented as examples only but can be modified in various ways within the scope of protection defined by the appended patent claims.

What is claimed is:

1. A compound, having a structural as follows: 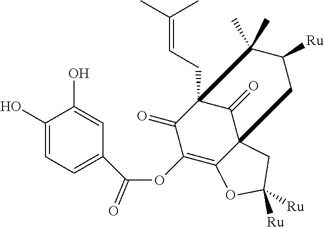
wherein R₁₀ is —C═CCH₃CH₃ or 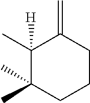
and R₁₁ is —H and R₁₂ is —CH₂OH, or R₁₁ and R₁₂ together with the carbon atom to which they attached form the group of: 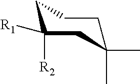
wherein R1 is independently —H, —OH or Me-32 and R2 is —OH or Me-32.

2. The compound according to claim 1, which is Paucinone A, wherein R₁₀ is —C═CCH₃CH₃ and R₁₁ and R₁₂ together with the carbon atom to which they attached form the group of: 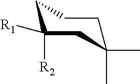
wherein R, is Me-32 and R₂ is —OH.

3. The compound according to claim 1, which is Paucinone B, wherein R₁₀ is —C═CCH₃CH₃ and R₁₁ and R₁₂ together with the carbon atom to which they attached form the group of: 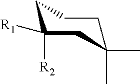
wherein R₂ is Me-32 and R, is —OH.

4. The compound according to claim 1, which is Paucinone C, wherein R₁₀ is —C═CCH₃CH₃ and R₁₁, and R₁₂ together with the carbon atom to which they attached form the group of: 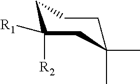
wherein R, is —H and R₂ is —OH.

5. The compound according to claim 1, which is Paucinone D, wherein R₁₁ is —H,
R₁₂ is —CH₂OH, R₁₀ is

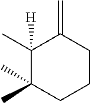

6. A method of inducing apoptosis in tumor cells, comprising a step of administering to a mammalian subject having tumor cells a therapeutically effective amount of a compound of claim 1.

7. The method according to claim 6, wherein said mammalian subject is a human patient suffering from cancer.

8. A pharmacological composition, comprising a compound of claim 1 and a pharmaceutically acceptable carrier.

9. The pharmacological composition according to claim 8, wherein the pharmacological composition is formulated in a dosage form selected from a group consisting of tablet, capsule, and injection.
